(19) United States
(12) Patent Application Publication
(10) Pub. No.: US 2024/0173526 A1
(43) Pub. Date: May 30, 2024

(54) VENOUS CANNULAS WITH INFLATABLE BALLOONS

(71) Applicants: NATIONAL UNIVERSITY OF SINGAPORE, Singapore (SG); NATIONAL UNIVERSITY HOSPITAL (SINGAPORE) PTE LTD, Singapore (SG)

(72) Inventor: Theodoros KOFIDIS, Singapore (SG)

(21) Appl. No.: 18/553,197

(22) PCT Filed: Mar. 29, 2022
(86) PCT No.: PCT/SG2022/050177

§ 371 Date: Sep. 28, 2023

(30) Foreign Application Priority Data
Mar. 29, 2021 (SG) 10202103213Q

(51) Int. Cl. A61M 25/10; A61M 25/00

(52) U.S. Cl. CPC A61M 25/1011; A61M 25/0026; A61M 25/0029; A61M 25/0045; A61M 25/1002; A61M 2025/0037; A61M 2025/0039; A61M 2025/1052; A61M 2025/1086

(57) ABSTRACT

The present disclosure provides a cannula comprising: a first tube having a first distal end for placement in a first vein of a patient; a first central lumen extending longitudinally along the first tube; at least one first hole positioned on the first distal end, the at least one hole fluidly connected to the first central lumen; at least one first peripheral lumen extending longitudinally along the first tube; and at least one first inflatable balloon connected to the first tube, each of the at least one first inflatable balloon fluidly connected to one of the at least one first peripheral lumen. The cannula may further comprise a branch cannula. The present disclosure further provides a cannula for placement within a first vein, an atrium and a second vein of a patient.

### CROSS REFERENCE TO RELATED APPLICATIONS

This application claims the benefit of Singapore Application No. 10202103213Q, filed on 29 Mar. 2021, the disclosure of which is hereby incorporated in its entirety by reference herein.

### TECHNICAL FIELD

The present disclosure relates broadly to venous cannulas. In particular, the venous cannulas may be utilized for the establishment of cardiopulmonary bypass in minimally invasive as well as open heart surgeries. Advantageously, the venous cannulas allow for faster and less traumatic performance of heart surgery.

### BACKGROUND

Heart surgeries require cannulation of the aorta and the right side of the heart to establish cardiopulmonary bypass such that blood is diverted into a heart-lung machine which oxygenates and pumps the blood back into the body through the aorta, thus isolating the heart to allow the heart to be operated on. Cannulation of the right side of the heart may involve cannulation of the right atrium, or bicaval cannulation, which is the cannulation of the superior vena cava (SVC) and inferior vena cava (IVC) by introducing two venous cannulas separately into the SVC and IVC. Bicaval cannulation is required in cases involving the mitral valve, the tricuspid valve, congenital holes in the heart, or redo operations.

Bicaval cannulation can be carried out either through a median sternotomy, or through a transfemoral route. The cannulas used have a long shaft and multiple holes near the tip of the cannula for blood to enter into the cannula to be diverted to the heart-lung machine. Once the cannulas have been inserted into the venae cavae, the SVC and IVC are dissected from their surroundings and a snare, also known as an occluding band, is passed around and tightened around each of the caval veins to cut off blood return into the heart. This is a time-consuming as well as risky maneuver, as it can result in injury of the caval veins or surrounding structures, with resulting devastating hemorrhage and other complications. This risk is even more pronounced in minimally invasive cardiac surgeries (MICS), where the heart is approached from a small right-lateral chest incision within a confined space, or in redo-surgery situations, where adhesions have formed.

It is thus desirable to provide venous cannulas that adequately divert blood from the heart while avoiding the dangerous digging around the SVC and IVC which could lead to perforations and massive hemorrhage.

### SUMMARY

There is provided according to an exemplary embodiment of the disclosure, a cannula comprising: a first tube having a first distal end for placement in a first vein of a patient; a first central lumen extending longitudinally along the first tube; at least one first hole positioned on the first distal end, the at least one hole fluidly connected to the first central lumen; at least one first peripheral lumen extending longitudinally along the first tube; and at least one first inflatable balloon connected to the first tube, each of the at least one first inflatable balloon fluidly connected to one of the at least one first peripheral lumen. Optionally, the least one first inflatable balloon is adapted to occlude the first central lumen and the first vein of the patient when inflated. Optionally, the at least one first inflatable balloon is positioned at a tip of the first tube within the first central lumen. Optionally, the first tube comprises one or more tube segments, the one or more tube segments connected to each other at least one beam. Optionally, each of the at least one first inflatable balloon is wrapped around each of the at least one beam. Optionally, the cannula is coated by one or more layers of drug selected from an anticoagulant drug, anti-inflammatory drug, anti-thrombogenic agent and mixture thereof.

In an exemplary embodiment of the disclosure, the at least one first inflatable balloon is wrapped around the first distal end of the tube, the at least one first inflatable balloon adapted to occlude the first vein of the patient when inflated.

In an exemplary embodiment of the disclosure, the cannula further comprises a branch cannula, the branch cannula comprising: a second tube having a second distal end for placement in a second vein of the patient; a second central lumen extending longitudinally along the second tube, the second central lumen fluidly connected to the first central lumen; at least one second hole positioned on the second distal end, the at least one second hole fluidly connected to the second central lumen; at least one second peripheral lumen extending longitudinally along the second tube; and at least one second inflatable balloon connected to the second tube, each of the at least one second inflatable balloon fluidly connected to one of the at least one second peripheral lumen. Optionally, the at least one second inflatable balloon is wrapped around the second distal end of the second tube, the at least one second inflatable balloon adapted to occlude the second vein of the patient when inflated. Optionally, the at least one first inflatable balloon or the at least one second inflatable balloon is a serrated inflatable balloon.

There is further provided according to an exemplary embodiment of the disclosure, a cannula comprising: a tube having a distal end for placement in a first vein, an atrium, and a second vein of a patient; a central lumen extending longitudinally along the tube; at least one peripheral lumen extending longitudinally along the tube; and a first and a second inflatable balloon connected to the tube, the first and second inflatable balloon each fluidly connected to one of the at least one peripheral lumen. Optionally, the first inflatable balloon and the second inflatable balloon are interspersed with an intra-atrial region. Optionally, the intra-atrial region is solid. Optionally, the size of the first and second inflatable balloons is the same. Optionally, the distance between the first and second inflatable balloons is between 6 to 14 cm. Optionally, the cannula is coated by one or more layers of drug selected from an anticoagulant drug, anti-inflammatory drug, anti-thrombogenic agent and mixture thereof. Optionally, the first or second inflatable balloon is a serrated inflatable balloon.

In an exemplary embodiment of the disclosure, the cannula further comprises a third hole in the intra-atrial region, the third hole connected to one of the at least one peripheral lumen extending longitudinally along the tube.

### BRIEF DESCRIPTION OF THE DRAWINGS

In order for the present disclosure, to be better understood and for its practical applications to be appreciated, the following Figures are provided and referenced hereafter. It should be noted that the Figures are given as examples only and in no way limit the scope of the invention.

FIGS. 1A and 1B schematically illustrate a cannula for cannulation of a vein, before and after at least one inflatable balloon has been inflated, in accordance with some embodiments of the present disclosure;

FIG. 1C schematically illustrates wire coiling spring within a cannula, in accordance with some embodiments of the present disclosure;

FIGS. 2A and 2B schematically illustrate a first alternative cannula for cannulation of a vein before and after at least one inflatable balloon has been inflated, in accordance with some embodiments of the present disclosure. Such embodiment is similar to the embodiments of FIGS. 1A and 1B except the first alternative cannula may have differing dimensions from the embodiments of FIGS. 1A and 1B;

FIGS. 3A and 3B schematically illustrate a second alternative cannula for cannulation of two veins before and after at least one inflatable balloon has been inflated, in accordance with some embodiments of the present disclosure;

FIGS. 4A and 4B schematically illustrate a third alternative cannula for cannulation of two veins before and after at least one inflatable balloon has been inflated, in accordance with some embodiments of the present disclosure. Such embodiment is similar to the embodiments of FIGS. 3A and 3B except the third alternative cannula may have differing dimensions from the embodiments of FIGS. 3A and 3B, the inflatable balloons may be placed in different positions, and the number of holes may differ;

FIGS. 5A and 5B schematically illustrate a fourth alternative cannula before and after at least one inflatable balloon has been inflated, in accordance with some embodiments of the present disclosure;

FIG. 5C is a perspective view of the fourth alternative cannula before the at least one inflatable balloon has been inflated, in accordance with some embodiments of the present disclosure;

FIG. 5D schematically illustrates the fourth alternative cannula within a heart of a patient after at least one inflatable balloon has been inflated, in accordance with some embodiments of the present disclosure;

FIG. 6 schematically illustrates an alternative distal end of fourth alternative cannula, further comprising a cardioplegia delivery catheter, in accordance with some embodiments of the present disclosure;

FIGS. 7A and 7B schematically illustrate a longitudinal cross-section of an inflatable balloon on a cannula adapted to occlude a vein before and after inflation, in accordance with some embodiments of the present disclosure;

FIGS. 8A to 8C schematically illustrate inflatable balloons on a cannula adapted to occlude a vein and a central lumen of the cannula, in accordance with some embodiments of the present disclosure;

FIG. 9 schematically illustrates an alternative embodiment of inflatable balloons on a cannula adapted to occlude a vein and a central lumen of the cannula, in accordance with some embodiments of the present disclosure; and

FIGS. 10A and 10B schematically illustrate outward and inward serrated inflatable balloons, in accordance with some embodiments of the present disclosure.

Identical or duplicate or equivalent or similar structures, elements, or parts that appear in one or more drawings are generally labeled with the same reference numeral, optionally with an additional letter or letters to distinguish between similar entities or variants of entities and may not be repeatedly labeled and/or described. References to previously presented elements are implied without necessarily further citing the drawing or description in which they appear.

### DETAILED DESCRIPTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, use of the conjunction “or” as used herein is to be understood as inclusive (any or all of the stated options).

In many respects the modifications of the various figures resemble those of preceding modifications and the same reference numerals followed by subscripts “a”, “b”, “c”, and “d” designate corresponding parts.

With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

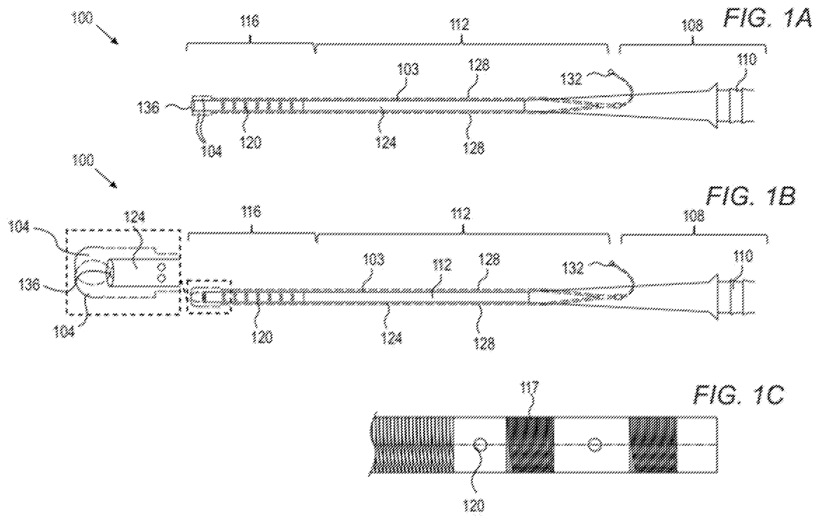

FIGS. 1A and 1B schematically illustrate a cannula 100 for cannulation of a vein, before and after at least one inflatable balloon 104 has been inflated, respectively, in accordance with some embodiments of the present disclosure. FIG. 1C schematically illustrates wire coiling spring 117 within the cannula 100, in accordance with some embodiments of the present disclosure. Cannula 100 may be inserted into a vein of a patient during a procedure. Although the term “cannula” is used in this disclosure, this may refer to any tube, catheter, or similar device. Cannula 100 may comprise a tube 103, cannula 100 comprising a proximal end 108, a main body 112 and a distal end 116. The cannula 100 main body 112 walls are preferably constructed of a flexible material which would not allow kinking and would preferably have a thickness of between 1.0 mm and 3.0 mm, and more preferably 1.5 mm. It is to be understood that other thickness not stated above but between the above range may also be used. In some embodiments, cannula 100 main body 112 walls are constructed of non-DEHP PVC (di-2-ethylhexyl phthalate polyvinyl chloride) or silicon, etc. The distal end of a cannula is understood herein to be the end of the cannula inserted into a patient during a procedure. In some embodiments, cannula 100 may be adapted to be positioned within an SVC of a patient. Cannula 100 may be shaped such that the proximal end 108 may have a length of between 8 cm and 15 cm, and preferably 11.5 cm, and a diameter of between 10 mm and 15 mm, the diameter gradually decreasing to a diameter of between 5 mm and 8.3 mm, and preferably 7.7 mm, at the main body 112 of cannula 100. In some embodiments, cannula 100 may have a distance of between 60 cm and 90 cm, and preferably 72.5 cm, from proximal end 108 to distal end 116 such that distal end 116 cannula 100 may be guided into and positioned within an SVC of a patient and proximal end 108 may be guided, positioned, and clamped. Preferably, cannula 100 may be reinforced with wire coiling spring 117 to prevent kinking of the cannula (see FIG. 1C). In some embodiments, wire coiling spring 117 may comprise one or more segments. In some embodiments, when three or more segments of wire coiling spring 117 are present, the distance between the adjacent segments of the wire coiling spring may be the same or varied. In some embodiments, wire coiling spring 117 may be made of stainless steel.

In some embodiments of the present disclosure, tube 103 of cannula 100 may enclose a central lumen 124 running longitudinally within tube 103. Tube 103 of cannula 100 may further comprise a plurality of holes 120 at distal end 116, the plurality of holes 120 fluidly connected to central lumen 124. Preferably, the plurality of holes 120 are provided perpendicular to the direction of liquid flow within central lumen 124 of cannula 100. Optionally, the plurality of holes 120 are bevelled towards the direction of liquid flow within central lumen 124 of cannula 100. Preferably, the plurality of holes 120 and central lumen 124 are adapted to conduct blood from a patient's vein to an external reservoir through proximal end 108 of cannula 100. Proximal end 108 of cannula 100 may have a connection site 110 for connection to a tubing connected to the external reservoir. Connection site 110 may have a diameter of between 8 mm and 15 mm, and preferably 12.5 mm. An example of an external reservoir is a heart-lung machine or extracorporeal membrane oxygenation reservoir (ECMO reservoir). In some embodiments, the position (or placement or arrangement) of holes 120, number of holes 120, diameter of holes 120 and diameter of the central lumen 124 of cannula 100 designed for an SVC may be adjusted and changed accordingly so that blood from the SVC flows through central lumen 124 of cannula 100 without interruption during a medical procedure at a flow rate of between 2 L/minute and 6 L/minute, and preferably 3 L/minute to sufficiently conduct blood flow the SVC. According to one embodiment, there may be 2 to 6 holes 120 disposed within 5 cm from a tip 136 of cannula 100 and, the diameter of central lumen 124 may range between 2.5 mm to 5.3 mm.

In some embodiments of the present disclosure, cannula 100 may comprise at least one inflatable balloon 104, the at least one inflatable balloon 104 is fluidly connected to at least one perimeter lumen 128 within tube 103 running longitudinally through cannula 100, the at least one perimeter lumen 128 is connected to a three-way connector 132 proximate to the proximal end 108 of cannula 100. The at least one inflatable balloon 104 may be positioned at any position along cannula 100. The at least one inflatable balloon 104 may comprise a flexible or pliable material such as, but not limited to, PVC, natural or synthetic rubbers, elastomers, vinyl plastisol, acrylic polyesters, rubbers, and other polymers or materials with similar resilience and pliability qualities. As illustrated in FIGS. 1A and 1B, the at least one inflatable balloon 104 may be positioned at the tip 136 of distal end 116 such that the at least one inflatable balloon 104 occludes the vein of the patient (not shown) as well as the central lumen 124 within cannula 100 when inflated, thereby creating a bloodless field for an operation surgeon especially during minimally invasive heart valve surgery. An operator may, using a guide sheath, with or without the assistance of imaging devices such as an ultrasound, insert distal end 116 of cannula 100 into the right internal jugular vein of the patient and guide or navigate distal end 116 of cannula 100 to the heart of the patient through the SVC until the at least one inflatable balloon 104 is positioned just before a junction where the SVC and the right atrium meet such that when the at least one inflatable balloon 104 is inflated, venous blood flow in the SVC is occluded without the need for an external clamp and thus advantageously preventing any external injury to the vessel. In some embodiments, cannula 100 may be partly or fully coated with one or more layers of drug to prevent or at least reduce blood clotting, bleeding risk and/or vessel muscle damage. Additionally, the coating may provide resistance to biofilm and pathogen adhesion. When cannula 100 is coated by one or more layers of drug, distal end 116 (including the at least one inflatable balloon 104), main body 112, proximal end 108, and central lumen 124 may be coated by one or more layers of drug. In some embodiments, the drug may be an anticoagulant drug, anti-inflammatory drug, anti-thrombogenic agent or mixture thereof. In some embodiments, the drug may be factor XII inhibitors or phosphoinositide 3-kinase inhibitors. Non-limiting examples of the anticoagulant drug include heparin, prostaglandins, enoxaparin, dalteparin, nadroparin, tinzaparin, warfarin, rivaroxaban, dabigatran, apixaban and parylene. In some embodiments, the anti-inflammatory drug may be a non-steroidal anti-inflammatory drug (NSAID) including, but not limited to, ibuprofen, naproxen, celecoxib, etoricoxib and diclofenac. Non-limiting examples of the anti-thrombogenic agent include low protein-binding polymeric coatings, tethered liquid perfluorocarbon (TLP) coating, fibronectin, collagen IV, phosphorylcholine and albumin-binding coating. The low protein-binding polymeric coatings may be zwitterionic hydrophilic coatings including polymers of sulfobetaine and polymers of carboxybetaine. The drug coated on the cannula, such as heparin coating, may alter surface properties of the cannula (i.e. lower friction or enhance lubricity), thereby facilitating the insertion of the cannula to the right internal jugular vein of the patient and guiding or navigating distal end of cannula to the heart of the patient, while providing hemocompatibility properties. The one or more layers of drug may be a hydrophilic coating. The coating process may be undertaken using a known method for example by a dip coating method in which the cannula is first immersed or submerged in a solution containing coating material comprising drug followed by drying to remove excess of the coating material. The coating process may be repeated to form two or more layers of different drugs. In some embodiments, the coating materials suitable for the above purpose may have at least one of the following properties: biocompatible, biostable, thermally stable, blood-compatible, resistance to biofilm and pathogen adhesion, ability to repel platelets, proteins, cells or other fouling materials.

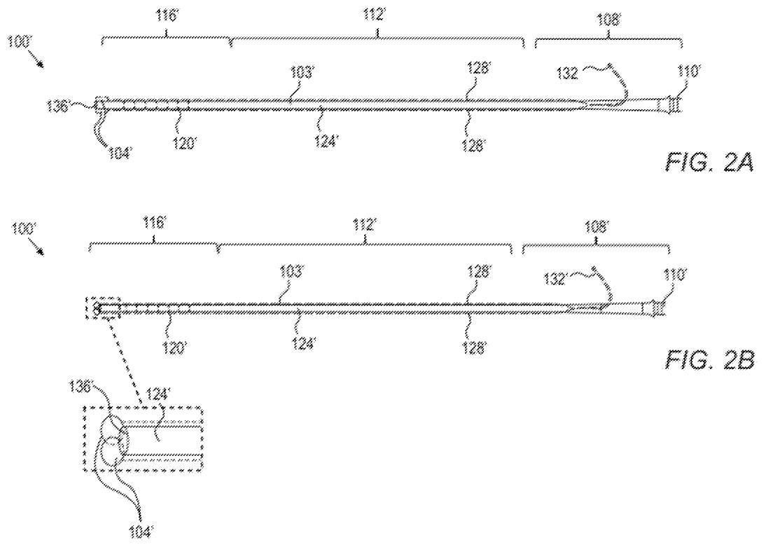

FIGS. 2A and 2B schematically illustrate a first alternative cannula 100′ for cannulation of a vein before and after at least one inflatable balloon 104′ has been inflated, respectively, in accordance with some embodiments of the present disclosure. Such embodiment is similar to the embodiments of FIGS. 1A and 1B except the first alternative cannula 100′ may have differing dimensions from the embodiments of FIGS. 1A and 1B. Cannula 100′ is shaped such that a proximal end 108′ and main body 112′ have a similar diameter of between 5 mm and 8.3 mm, and preferably 7.7 mm. In some embodiments, cannula 100′ may have a distance of between 60 cm and 90 cm, and preferably 72.5 cm, from proximal end 108′ to distal end 116′ such that distal end 116′ of cannula 100′ may be positioned within an IVC of a patient and proximal end 108′ may be positioned and clamped. The internal dimensions of the cannula 100′ of FIGS. 2A and 2B may be similar to those of the cannula 100 of FIGS. 1A, 1B. The materials used to fabricate the cannula 100′ of FIGS. 2A and 2B may be similar to those of the cannula 100 of FIGS. 1A, 1B.

In some embodiments of the present disclosure, a plurality of holes 120′ within tube 103′ at distal end 116′ are adapted to conduct blood from a patient's vein to an external reservoir through proximal end 108′ of cannula 100′. The position (or placement or arrangement) of the plurality of holes 120′, number of the plurality of holes 120′, diameter of the plurality of holes 120′ and diameter of the central lumen 124′ of cannula 100′ designed for an IVC may be adjusted and changed accordingly so that blood from the IVC flows through central lumen 124′ of cannula 100′ without interruption during a medical procedure at a flow rate of between 2 L/minute and 6 L/minute, and preferably 3 L/minute to sufficiently conduct blood from the IVC. According to one embodiment, there may be 10 to 14 holes 120′ provided within 15 cm from a tip 136′ of cannula 100′, and the diameter of central lumen 124′ may range between 2.5 mm and 5.3 mm.

In some embodiments of the present disclosure, an operator may, using a guide sheath, with or without the assistance of imaging devices, such as ultrasound, insert distal end 116′ of cannula 100′ into a right femoral vein and guide or navigate distal end 116′ of cannula 100′ to the heart through the IVC until the at least one inflatable balloon 104′ is positioned just before a junction where the IVC and the right atrium meet such that when the at least one inflatable balloon 104′ is inflated, venous blood flow in the IVC is occluded without the need for an external clamp and thus advantageously preventing any external injury to the vessel.

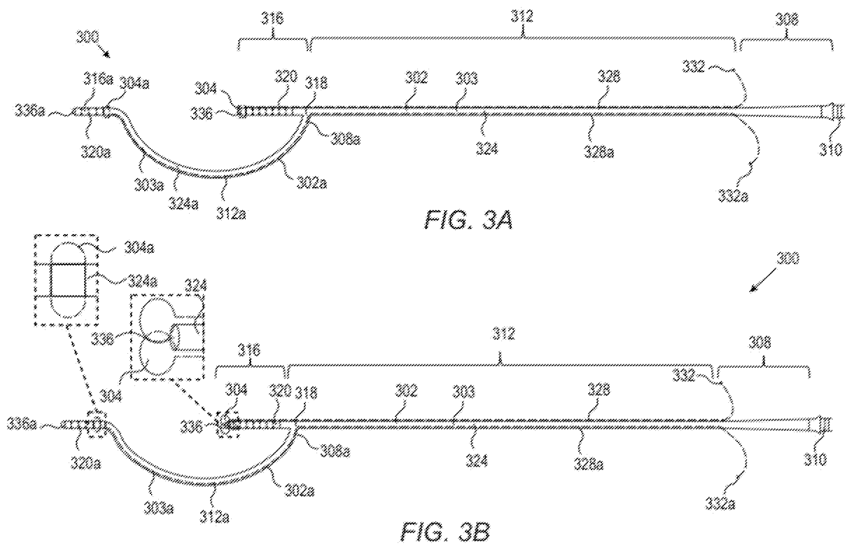

FIGS. 3A and 3B schematically illustrate a second alternative cannula 300 for cannulation of two veins before and after at least one inflatable balloon 304 has been inflated, respectively, in accordance with some embodiments of the present disclosure. Cannula 300 may comprise a main cannula 302 comprising a tube 303 and a branch cannula 302a comprising a tube 303a. In some embodiments, main cannula 302 and branch cannula 302a may be connected to each other. In other embodiments, main cannula 302 may comprise a cannula lumen (not shown) with an exit opening (not shown), the cannula lumen adapted to receive a separate branch cannula 302a that exits through the exit opening. Main cannula 302 and branch cannula 302a may individually be inserted into a vein of a patient during a procedure. Advantageously, main cannula 302 may be inserted into an IVC of the patient while branch cannula 302a may be inserted into an SVC of the patient. As in cannula 100′, main cannula 302 comprises a proximal end 308, a main body 312 and a distal end 316. Main cannula 302 may be shaped such that the proximal end 308 and main body 312 may have a similar diameter of between 5 mm and 8.3 mm, and preferably 7.7 mm. In some embodiments, main cannula 302 may have a distance between 60 cm and 90 cm, and preferably 72.5 cm, from proximal end 308 to distal end 316 such that distal end 316 of main cannula 302 may be positioned within an IVC of a patient and proximal end 308 may be positioned and clamped.

In some embodiments of the present disclosure, tube 303 of main cannula 302 may enclose a central lumen 324 running longitudinally within tube 303, and tube 303a of branch cannula 302a may enclose a central lumen 324a running longitudinally within tube 303a. Central lumen 324 of main cannula 302 and central lumen 324a of branch cannula 302a may be fluidly connected. Branch cannula 302a may, similar to main cannula 302, have a distal end 316a, a main body 312a and a proximal end 308a, proximal end 308a of branch cannula 302a connected to main cannula 302 at a junction 318 along main body 312 of main cannula 302 proximate to distal end 316 of main cannula 302. In some embodiments, junction 318 may be located between 30 cm to 50 cm from distal end 316 of main cannula 302. In some embodiments, branch cannula 302a may have a distance between 30 cm and 50 cm, and preferably 40 cm, from its distal end 316a to junction 318.

In some embodiments of the present disclosure, tube 303a of branch cannula 302a may further comprise a plurality of holes 320a at distal end 316a, the holes 320a fluidly connected to central lumen 324a. The holes 320a and central lumen 324a may be adapted to conduct blood from a patient SVC through central lumen 324 of main cannula 302 to an external reservoir. In some embodiments, there may be 4 to 6 holes 320a within about 5 cm from tip 336a of branch cannula 302a. Central lumen 324a of branch cannula 302a may have a diameter of between 5.0 mm and 8.3 mm, and preferably 7.7 mm. The position (or placement or arrangement) of holes 320a, number of holes 320a, diameter of holes 320a and diameter of the central lumen 324a of branch cannula 302a designed for an SVC may be adjusted and changed accordingly so that blood from the SVC flows through central lumen 324a of branch cannula 302a without interruption during a medical procedure at a flow rate of between 2 L/minutes and 6 L/minutes, and preferably 3 L/minutes to sufficiently conduct blood from the SVC. According to one embodiment, central lumen 324a may have a diameter of between 2.5 mm and 5.3 mm.

In some embodiments of the present disclosure, tube 303 of main cannula 302 may further comprise a plurality of holes 320 at distal end 316, the holes 320 fluidly connected to central lumen 324. The holes 320 may be adapted to conduct blood from a patient IVC into central lumen 324 of main cannula 302. Central lumen 324 may be adapted to receive blood from the IVC of a patient through holes 320, as well as blood from the SVC of the patient through central lumen 324a of branch cannula 320a. Central lumen 324 of main cannula 302 may be adapted to conduct blood to an external reservoir at proximal end 308. Proximal end 308 of main cannula 302 may have a connection site 310 for connection to a tubing connected to the external reservoir. Connection site 310 may have a diameter of between 8 mm and 15 mm, and preferably 12.5 mm. The position (or placement or arrangement) of holes 320, number of holes 320 and diameter of holes 320 may be adjusted and changed accordingly to ensure that blood flows into central lumen 324 of main cannula 302 at a flow rate of between 2 L/minute and 6 L/minute, and preferably 3 L/minute to sufficiently conduct blood from the IVC. In accordance with one embodiment there may be 10 to 14 holes 320 within 15 cm from tip 336 of main cannula 302. Central lumen 324 of main cannula 302 may have a diameter of between 2.5 mm and 5.3 mm, and preferably 4.7 mm, to adequately conduct the volume of blood flowing from both the SVC and IVC of the patient. The diameter of central lumen 324 may be adjusted and changed accordingly to ensure that blood from the IVC and SVC flows through central lumen 324 without interruption during a medical procedure at a flow rate of between 2 L/minute and 6 L/minute, and preferably 3 L/minute to sufficiently conduct blood from both the SVC and IVC. According to one embodiment, central lumen 324 may have a diameter of between 2.5 mm and 5.3 mm.

In some embodiments of the present disclosure, main cannula 302 may comprise at least one inflatable balloon 304, the at least one inflatable balloon 304 fluidly connected to at least one perimeter lumen 328 within tube 303 running longitudinally through main cannula 302, the at least one perimeter lumen 328 connected to a first three-way connector 332 proximate to the proximal end 308 of main cannula 302. Preferably, the at least one inflatable balloon 304 may be positioned at a tip 336 of distal end 316 of main cannula 302 such that the at least one inflatable balloon 304 occludes the IVC of the patient (not shown) as well as the central lumen 324 within main cannula 302 when inflated.

In some embodiments of the present disclosure, branch cannula 302a may comprise at least one inflatable balloon 304a, the at least one inflatable balloon 304a fluidly connected to at least one perimeter lumen 328a within tube 303a running longitudinally through branch cannula 302a and main cannula 302, the at least one perimeter lumen 328a connected to a second three-way connector 332a proximate to the proximal end 308 of main cannula 302. Preferably, the at least one inflatable balloon 304a may be positioned between a plurality of holes 320a and main body 312a of branch cannula 302a. Preferably, the at least one inflatable balloon 304a occludes the SVC of the patient (not shown) without occluding the central lumen 324a within branch cannula 302a when inflated.

In some embodiments of the present disclosure, an operator may insert distal end 316 of main cannula 302 into the right femoral vein and navigate distal end 316 of main cannula 302 to the heart through the IVC until the at least one inflatable balloon 304 is positioned just before a junction where the IVC and the right atrium meet such that when the at least one inflatable balloon 304 is inflated, venous blood flow in the IVC is advantageously occluded without the need for an external clamp and thus preventing any external injury to the vessel. The operator may further manipulate branch cannula 302a outside the body of the patient and then guide branch cannula 302a back into the SVC by inserting distal end 316a of branch cannula 302a through a right atrial appendage until the at least one inflatable balloon 304a is positioned just before a junction where the SVC and the right atrium meet such that when the at least one inflatable balloon 304a is inflated, venous blood flow in the SVC is advantageously occluded without the need for an external clamp and thus preventing any external injury to the vessel.

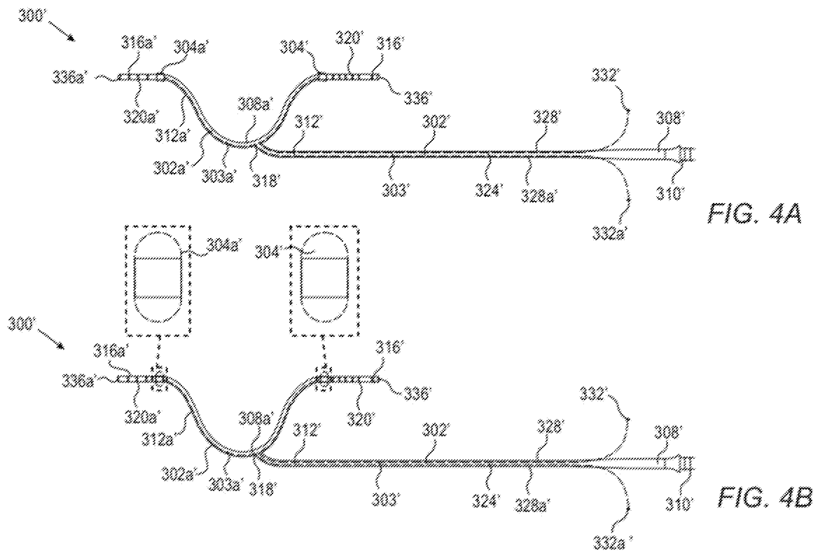

FIGS. 4A and 4B schematically illustrate a third alternative cannula 300′ for cannulation of two veins before and after at least one inflatable balloon 304′ has been inflated, respectively, in accordance with some embodiments of the present disclosure. Such embodiment is similar to the embodiments of FIGS. 3A and 3B except the third alternative cannula 300′ may have differing dimensions from the embodiments of FIGS. 3A and 3B, the inflatable balloons may be placed in different positions, and the number (or placement or arrangement) of holes may differ. In third alternative cannula 300′, junction 318′ may be located equidistant between distal end 316′ of main cannula 302′ and distal end 316a′ of branch cannula 302a′. In some embodiments, junction 318′ may be positioned between 15 cm to 25 cm from distal end 316′ of main cannula 302′ and distal end 316a′ of branch cannula 302a′. In some other embodiments, the distance between distal end 316′ of main cannula 302′ and distal end 316a′ of branch cannula 302a′ may differ to enable easier cannulation of the SVC and IVC and to prevent unexpected or expected decrease in venous flows to prevent patient exsanguination. In some embodiments, junction 318′ may be positioned between 30 cm to 50 cm from distal end 316′ of main cannula 302′ and may be positioned between 50 cm to 70 cm distal end 316a′ of branch cannula 302a′.

In some embodiments of the present disclosure, both main cannula 302′ and branch cannula 302a′ of third alternative cannula 300′ may further comprise 4 to 6 holes 320′ and 320a′ within 20 cm from tip 336′ and 336a′ respectively. In some embodiments of the present disclosure, at least one inflatable balloon 304a′ may be positioned between a plurality of holes 320a′ and main body 312a′ of branch cannula 302a. Preferably, the at least one inflatable balloon 304a′ when inflated occludes the SVC of the patient (not shown) without occluding the central lumen 324a′ within branch cannula 302a′ when inflated. In some embodiments of the present disclosure, at least one inflatable balloon 304′ may be positioned between holes 320′ and main body 312′ of main cannula 302′. Preferably, the at least one inflatable balloon 304′ when inflated occludes the IVC of the patient (not shown) without occluding the central lumen 324′ within main cannula 302′ when inflated. In some embodiments, the diameter of central lumen 324′ of main cannula 302′ may be between 2.5 mm and 5.3 mm, and the diameter of central lumen 324′ of branch cannula 302′ may be between 2.5 mm and 5.3 mm thus advantageously allowing uninterrupted blood flow during a surgical procedure and preventing exsanguination.

In some embodiments of the present disclosure, during surgery, an operator may insert distal end 316′ of main cannula 302′ through a right atrial appendage into the IVC until the at least one inflatable balloon 304′ is positioned just before a junction where the IVC and the right atrium meet such that when the at least one inflatable balloon 304′ is inflated, venous blood flow in the IVC is advantageously occluded without the need for an external clamp and thus preventing any external injury to the vessel. The operator may further insert distal end 316a′ of branch cannula 302a′ through a right atrial appendage into the SVC until the at least one inflatable balloon 304′ is positioned just before a junction where the SVC and the right atrium meet such that when the at least one inflatable balloon 304a is inflated, venous blood flow in the SVC is occluded without the need for an external clamp and thus preventing any external injury to the vessel.

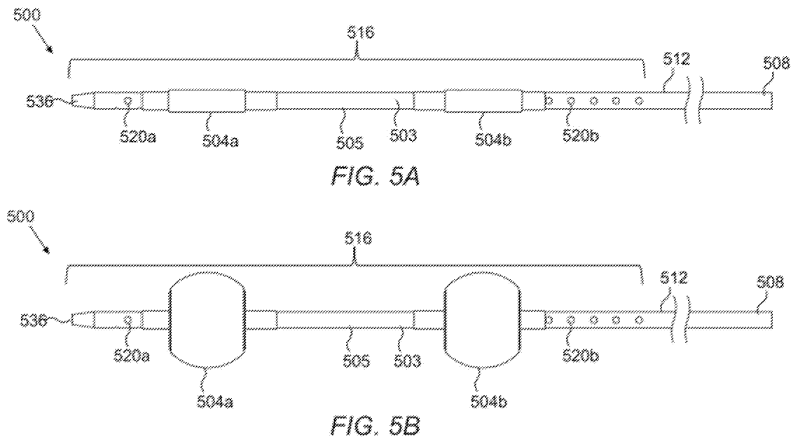

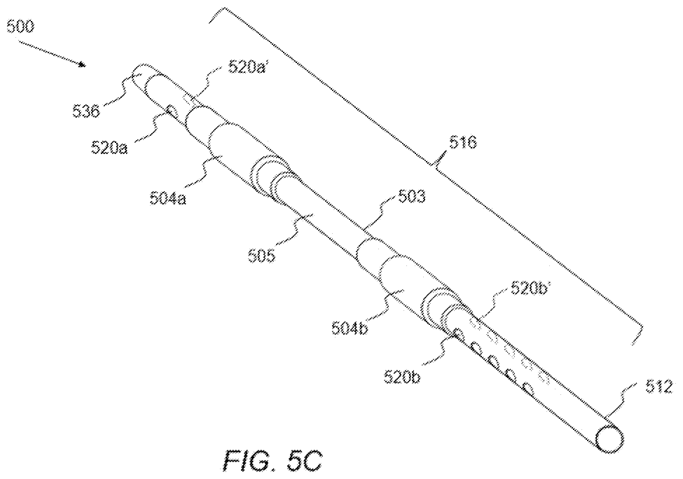

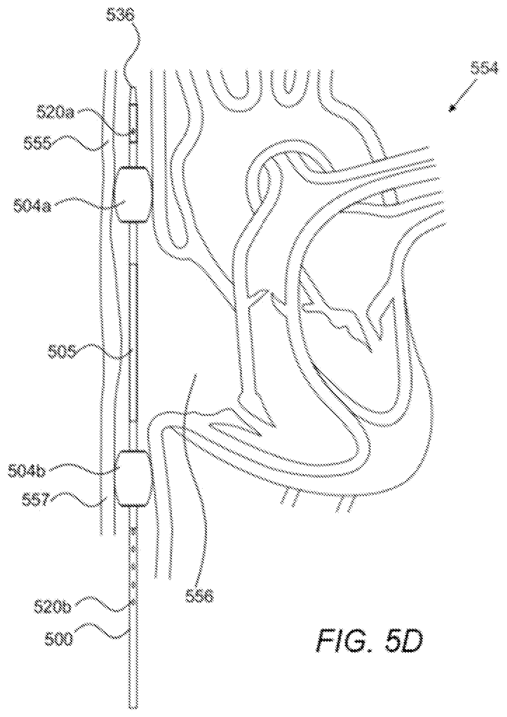

FIGS. 5A and 5B schematically illustrate a fourth alternative cannula 500 before and after at least one inflatable balloon 504 has been inflated, respectively, in accordance with some embodiments of the present disclosure. FIG. 5C is a perspective view of the fourth alternative cannula 500 before the at least one inflatable balloon 504 has been inflated, in accordance with some embodiments of the present disclosure. FIG. 5D schematically illustrates the fourth alternative cannula 500 within a heart 554 of a patient after at least one inflatable balloon 504 has been inflated, in accordance with some embodiments of the present disclosure. As in cannula 100, cannula 500 comprises a tube 503, cannula 500 comprising a proximal end 508, a main body 512 and a distal end 516. In some embodiments, cannula 500 may be inserted, guided and positioned to a patient's SVC 555, right atrium 556, and IVC 557. Cannula 500 may be shaped such that main body 512 may have a diameter from 18 Fr to 29 Fr, such as 18 Fr, 19 Fr, 20 Fr, 21 Fr, 22 Fr, 23 Fr, 24 Fr, 25 Fr, 26 Fr, 27 Fr, 28 Fr and 29 Fr. In some embodiments, cannula 500 has a diameter of 23 Fr. In some embodiments, when the diameter of cannula 500 is 23 Fr, such cannula may be used in conjunction with an introducer of 19 Fr. In some embodiments, cannula 500 has a diameter of 29 Fr. In some embodiments, when the diameter of cannula 500 is 29 Fr, such cannula may be used in conjunction with an introducer of 25 Fr. “Fr” refers to French scale or French gauge, wherein 3 Fr is equivalent to about 1 mm. In some embodiments, cannula 500 may have a distance between 60 cm and 90 cm, and preferably 72.5 cm, from proximal end 508 to distal end 516 such that distal end 516 of cannula 500 may be positioned within a patient's SVC, right atrium, and IVC and proximal end 508 may be positioned and clamped.

In some embodiments of the present disclosure, cannula 500 may, as in cannula 100, comprise at least one inflatable balloon 504, the at least one inflatable balloon 504 fluidly connected to at least one perimeter lumen (not shown) within tube 503 running longitudinally through cannula 500. As illustrated in FIGS. 5A, 5B, 5C and 5D, catheter 500 may comprise inflatable balloons 504a and 504b at distal end 516. Inflatable balloons 504a and 504b interspersed with an intra-atrial region 505. In some embodiments, inflatable balloons 504a and 504b are each fluidly connected to a separate perimeter lumen (not shown) so that an operator may selectively inflate inflatable balloons 504a and 504b. In some embodiments, there may be two or more inflatable balloons 504a and 504b, such that an operator may selectively inflate the two or more inflatable balloons 504a and 504b depending on the right atrium size of the patient. In some embodiments, the at least one perimeter lumen (not shown) may each be connected to a separate three-way connector (not shown) proximate to the proximal end 508, each three-way connector (not shown) further connected to a syringe (not shown). Optionally, each syringe (not shown) may be labelled so that the operator may easily select the inflatable balloon that they wish to inflate. The syringes may be labelled with text, or with colour. Preferably, inflatable balloon 504a is positioned between 3 cm and 7 cm, and preferably 5 cm, from a tip 536 of distal end 516 of cannula 500. Preferably, there is a distance of between 6 cm and 14 cm, such as 6 cm, 8 cm, 10 cm, 12 cm, and 14 cm between inflatable balloons 504a and 504b such that when cannula 500 is inserted into a heart of a patient, inflatable balloon 504a is positioned within the SVC 555 just before a junction where the SVC 555 and the right atrium 556 meet, and inflatable balloon 504b is positioned within the IVC 557 just before a junction where the IVC 557 and the right atrium 556 meet. The distance between the two inflatable balloons above may be used for the two inflatable balloons of different or identical size. When inflatable balloon 504a is inflated, inflatable balloon 504a occludes the SVC 555 and prevents blood from flowing from the SVC 555 to the right atrium 556. Instead, the blood flows into cannula 500. When inflatable balloon 504b is inflated, inflatable balloon 504b occludes the IVC 557 and prevents blood from flowing from the IVC 557 to the right atrium 556. Instead, the blood flows into cannula 500. In some embodiments, cannula 500 has a diameter of 23 Fr, the distance between two inflatable balloons (each for IVC and SVC, respectively) may be 6 cm, 8 cm, 10 cm, or 12 cm, whereby the size of the balloons for both IVC and SVC is 30 mm. In some embodiments, cannula 500 may be partly or fully coated with one or more layers of drug to prevent or at least reduce blood clotting, bleeding risk and/or vessel muscle damage. Additionally, the coating may provide resistance to biofilm and pathogen adhesion. When the cannula 500 is coated by one or more layers of drug, distal end 516 (including the inflatable balloons 504a and 504b), main body 512 and proximal end 508 may be coated by one or more layers of drug. In some embodiments, the drug may be an anticoagulant drug, anti-inflammatory drug, anti-thrombogenic agent or mixture thereof. In some embodiments, the drug may be factor XII inhibitors or phosphoinositide 3-kinase inhibitors. Non-limiting examples of the anticoagulant drug include heparin, prostaglandins, enoxaparin, dalteparin, nadroparin, tinzaparin, warfarin, rivaroxaban, dabigatran, apixaban and parylene. In some embodiments, the anti-inflammatory drug may be a non-steroidal anti-inflammatory drug (NSAID) including, but not limited to, ibuprofen, naproxen, celecoxib, etoricoxib and diclofenac. Non-limiting examples of the anti-thrombogenic agent include low protein-binding polymeric coatings, tethered liquid perfluorocarbon (TLP) coating, fibronectin, collagen IV, phosphorylcholine and albumin-binding coating. The low protein-binding polymeric coatings may be zwitterionic hydrophilic coatings including polymers of sulfobetaine and polymers of carboxybetaine. The drug coated on the cannula, such as heparin coating, may alter surface properties of the cannula (i.e. lower friction or enhance lubricity), thereby facilitating the insertion of the cannula to the right internal jugular vein of the patient and guiding or navigating distal end of cannula to the heart of the patient, while providing hemocompatibility properties. The one or more layers of drug may be a hydrophilic coating. The coating process may be undertaken using a known method for example by a dip coating method in which the cannula is first immersed or submerged in a solution containing coating material comprising drug followed by drying to remove excess of the coating material. The coating process may be repeated to form two or more layers of different drugs. In some embodiments, the coating materials suitable for the above purpose may have at least one of the following properties: biocompatible, biostable, thermally stable, blood-compatible, resistance to biofilm and pathogen adhesion, ability to repel platelets, proteins, cells or other fouling materials.

In some embodiments of the present disclosure, tube 503 of cannula 500 may, as in cannula 100, enclose a central lumen (not shown) running longitudinally within tube 503. Tube 503 may further comprise a plurality of holes 520 at distal end 516 of cannula 500, the holes 520 fluidly connected to central lumen (not shown), holes 520 and central lumen (not shown) adapted to conduct blood from a patient's vein to an external reservoir through proximal end 508 of cannula 500. In some embodiments, cannula 500 may comprise a plurality of holes 520a between tip 536 and inflatable balloon 504a to conduct blood from a patient's SVC 555 to an external reservoir. For example, there may be 2 to 4 holes 520a within a distance of 5 cm of tip 536. In some embodiment, as can be seen in FIG. 5C, there may be 2 holes (520a and 520a′, also known as multiple side holes) placed within the same distance from tip 536. In other embodiments, cannula 500 may comprise a plurality of holes 520b between inflatable balloon 504b and main body 512 of cannula 500 to conduct blood from a patient's IVC 557 to an external reservoir. For example, there may be 10 to 32 holes 520b within a distance of between 15 cm to 20 cm of inflatable balloon 504b. There may be 2 to 4 holes 520b positioned within the same distance of between 15 cm to 20 cm of inflatable balloon 504b. In some embodiment, as can be seen in FIG. 5C, there may be 10 holes (520b and 520b′) within a distance between 15 cm to 20 cm of inflatable balloon 504b. In some embodiment, there may be 12 or 14 holes (i.e. side holes 520b and 520b′) within the distance between 15 cm to 20 cm of inflatable balloon 504b. The 10, 12 or 14 holes may be placed in 5, 6 or 7 different regions, wherein each region may contain a pair of side holes 520b and 520b′ within the distance between 15 cm and 20 cm. In other embodiments, intra-atrial region 505 between inflatable balloon 504a and 504b is solid and devoid of any holes connecting to the central lumen (not shown). In some embodiments, intra-atrial region 505 between inflatable balloon 504a and 504b comprises a plurality of holes (not shown) connecting to the central lumen. In some embodiments, the position of holes 520, number of holes 520, diameter of holes 520 and diameter of the central lumen of cannula 500 may be adjusted and changed accordingly so that blood flows through the central lumen of cannula 500 at a flow rate of between 2 L/minute and 5 L/minute, and preferably 3 L/minute to sufficiently conduct blood from both the SVC 555 and IVC 557. In some embodiments, the diameter of the central lumen of cannula 500 may be similar to the diameter of central lumen 124 and 124′ of FIGS. 1A and 1B, allowing uninterrupted blood flow during a surgical procedure and preventing exsanguination.

In some embodiments of the present disclosure, tip 536 of cannula 500 may further comprise an intra-luminal vascular ultrasound (IVUS) microsensor (not shown) to evaluate a condition of a vein when cannula 500 is in use. The IVUS microsensor may be connected via microtubular electronic connection. Alternatively, the IVUS microsensor may be monitored by a remote accessed microchip. Alternatively, the IVUS may be connected via a nano-chip mechanism.

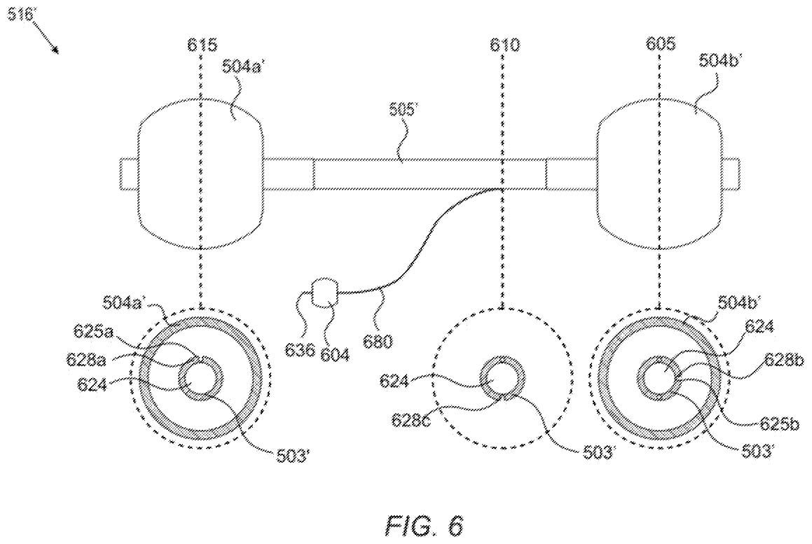

FIG. 6 schematically illustrates an alternative distal end 516′ of fourth alternative cannula 500, further comprising a cardioplegia delivery catheter 680, in accordance with some embodiments of the present disclosure. Cardioplegia delivery catheter 680 is adapted to deliver cardioplegia solution to the coronary sinus to induce heart arrest. Transverse cross-sectional cuts 605, 610 and 615 schematically illustrate lumens in cannula 500. Cannula 500 comprises a central lumen 624 running longitudinally within cannula 500 from tip 536 to proximal end 508. Cannula 500 may further comprise perimeter lumens 628a, 628b and 628c within tube 503′. Perimeter lumen 628b may terminate within inflatable balloon 504b′ and may comprise at least one opening 625b positioned within inflatable balloon 504b′ to fluidly connect perimeter lumen 628b to inflatable balloon 504b′, such that inflatable balloon 504b′ is inflated by air or liquid introduced through perimeter lumen 628b. Perimeter lumen 628a may terminate within inflatable balloon 504a′ and may comprise at least one opening 625a positioned within inflatable balloon 504a′ to connect perimeter lumen 628a to inflatable balloon 504a′, such that inflatable balloon 504a′ is inflated by air or liquid introduced through perimeter lumen 628a.

In some embodiments of the present disclosure, perimeter lumen 628c may comprise an opening 625c positioned along intra-atrial region 505′. Perimeter lumen 628c may have a diameter of between 0.5 mm and 1.5 mm, and preferably 1 mm, to accommodate or receive a cardioplegia delivery catheter 680.

In some embodiments of the present disclosure, cardioplegia delivery catheter 680 may deliver cardioplegia through a tip 636. Cardioplegia delivery catheter 680 may further comprise an inflatable balloon 604 near tip 636. An operator may insert cardioplegia delivery catheter 680 into perimeter lumen 628c of cannula 500 prior to insertion of cannula 500 into a patient. The operator may then navigate cannula 500 into a heart 554 of a patient and inflate inflatable balloons 504a′ and 504b′ to occlude SVC 555 and IVC 557 to isolate right atrium 556. The operator may, using forceps or tweezers or a similar instrument, navigate and insert cardioplegia delivery catheter 680 into a coronary sinus of the patient. The operator may then inflate inflatable balloon 604 of cardioplegia delivery catheter 680 to secure or fix the position of cardioplegia delivery catheter 680 within the coronary sinus. Finally, the operator may inject cardioplegia solution into a proximal end (not shown) of cardioplegia delivery catheter 680 to be delivered through tip 636 to the coronary sinus to arrest the heart.

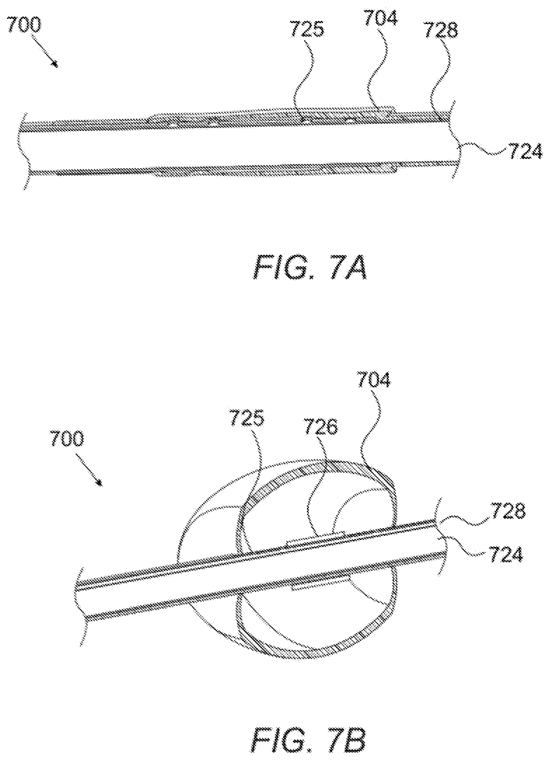

FIGS. 7A and 7B schematically illustrate a longitudinal cross-section of an inflatable balloon 704 on a cannula 700 adapted to occlude a vein before and after inflation, in accordance with some embodiments of the present disclosure. Cannula 700 may comprise a central lumen 724 and at least one perimeter lumen 728. Perimeter lumen 728 may be connected to a three-way connector (not shown).

In some embodiments of the present disclosure, cannula 700 may further comprise inflatable balloon 704 wrapped around cannula 700. Inflatable balloon 704 may be made of super elastic material, such as PVC, silicon, etc, that lays substantially flat against cannula 700 when deflated. Alternatively, cannula 700 may have a slight depression to accommodate inflatable balloon 704. Inflatable balloon 704 may be fluidly connected to perimeter lumen 728 by openings 725 along perimeter lumen 728, such that when air or liquid is introduced through a three-way connector into perimeter lumen 728, the air or liquid enters inflatable balloon 704 and inflates the same. Inflatable balloon 704 may be deflated when air or liquid is aspirated from perimeter lumen 728. Preferably, perimeter lumen 728 comprises between 2 to 4 openings 725, and preferably 4 openings 725. Inflatable balloon 704 may have a diameter of between 20 mm to 30 mm when inflated to occlude the vena cava of a patient. In some embodiments, saline or iodine-based contrast media may be used to inflate inflatable balloons 704.

In some embodiments of the present disclosure, cannula 700 may have two or more inflatable balloons 704, each inflatable balloon 704 fluidly connected to a separate perimeter lumen 728, such that an operator may selectively inflate a desired inflatable balloon 704. When cannula 700 has two or more inflatable balloons 704, the two or more inflatable balloons may be of the same or different size. If there are two or more inflatable balloons 704, the operator may employ a “kissing balloon” technique to confirm the optimal balloon inflation and luminal occlusion. The “kissing balloon” technique will modify the geometry of the two or more inflatable balloons 704, but other factors are also involved in the inflation process, including the balloon size, inflation pressure and deflation sequence. In some embodiments, cannula 700 may be partly or fully coated with one or more layers of drug to prevent or at least reduce blood clotting, bleeding risk and/or vessel muscle damage. Additionally, the coating may provide resistance to biofilm and pathogen adhesion. When the cannula 700 is coated by one or more layers of drug, central lumen 724 and inflatable balloons 704 may be coated by one or more layers of drug. In some embodiments, the drug may be an anticoagulant drug, anti-inflammatory drug, anti-thrombogenic agent or mixture thereof. In some embodiments, the drug may be factor XII inhibitors or phosphoinositide 3-kinase inhibitors. Non-limiting examples of the anticoagulant drug include heparin, prostaglandins, enoxaparin, dalteparin, nadroparin, tinzaparin, warfarin, rivaroxaban, dabigatran, apixaban and parylene. In some embodiments, the anti-inflammatory drug may be a non-steroidal anti-inflammatory drug (NSAID) including, but not limited to, ibuprofen, naproxen, celecoxib, etoricoxib and diclofenac. Non-limiting examples of the anti-thrombogenic agent include low protein-binding polymeric coatings, tethered liquid perfluorocarbon (TLP) coating, fibronectin, collagen IV, phosphorylcholine and albumin-binding coating. The low protein-binding polymeric coatings may be zwitterionic hydrophilic coatings including polymers of sulfobetaine and polymers of carboxybetaine. The drug coated on the cannula, such as heparin coating, may alter surface properties of the cannula (i.e. lower friction or enhance lubricity), thereby facilitating the insertion of the cannula to the right internal jugular vein of the patient and guiding or navigating distal end of cannula to the heart of the patient, while providing hemocompatibility properties. The one or more layers of drug may be a hydrophilic coating. The coating process may be undertaken using a known method for example by a dip coating method in which the cannula is first immersed or submerged in a solution containing coating material comprising drug followed by drying to remove excess of the coating material. The coating process may be repeated to form two or more layers of different drugs. In some embodiments, the coating materials suitable for the above purpose may have at least one of the following properties: biocompatible, biostable, thermally stable, blood-compatible, resistance to biofilm and pathogen adhesion, ability to repel platelets, proteins, cells or other fouling materials.

In some embodiments of the present disclosure, cannula 700 may further comprise a pressure sensor 726 wrapped around cannula 700 within an area surrounded by inflatable balloon 704. Pressure sensor 726 monitors the inflation and pressure of inflatable balloon 704 to advantageously prevent balloon or vessel wall rupture. In some embodiments, an area on cannula 700 proximate to inflatable balloon 704 may comprise radio-opaque wires to identify inflatable balloons 704 during a procedure. In some embodiments of the present, inflatable balloon 704 may be replaced with a region of super-elastic material (not shown) embedded with remote access nano-chip controllers (not shown), the remote access nano-chip controllers controlled by a remote control device (not shown) such that when the remote access nano-chip controllers are activated by the remote control device, the region of super-elastic material deforms and forms aneurysmal dilation of the region to occlude a vein of a patient.

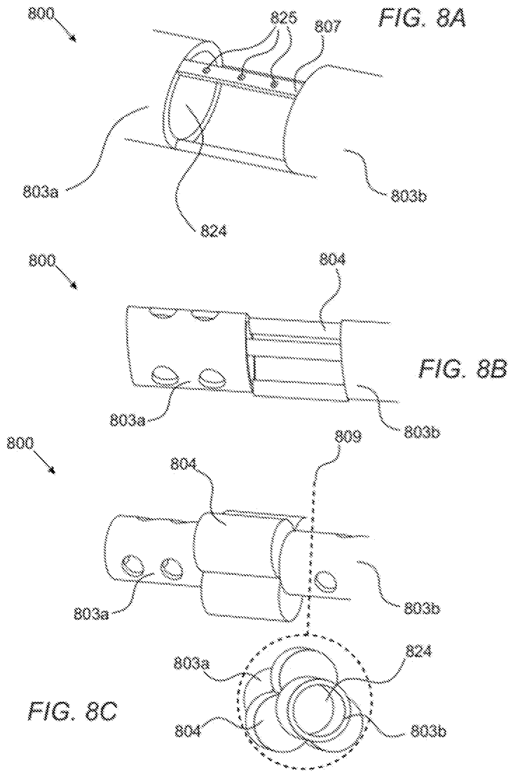

FIGS. 8A to 8C schematically illustrate inflatable balloons 804 on a cannula 800 adapted to occlude a vein and a central lumen of the cannula, in accordance with some embodiments of the present disclosure. Cannula 800 may comprise a tube 803 enclosing a central lumen 824. Tube 803 may be divided into a first tube segment 803a and a second tube segment 803b, first tube segment 803a and second tube segment 803b connected by at least one beam 807. In some embodiments, there may be 2 to 4, and preferably 3, beams 807. Beams 807 may have a length of between 3 cm to 6 cm, and preferably 4 cm. In some embodiments, cannula 800 may comprise at least one perimeter lumen (not shown), the at least one perimeter lumen running longitudinally from first tube segment 803a and through beam 807.

In some embodiments of the present disclosure, each of the at least one beam 807 may further comprise an inflatable balloon 804 wrapped around the beam 807. Inflatable balloon 804 may be fluidly connected to the perimeter lumen (not shown) running longitudinally through beam 807 by openings 825, such that when air or liquid is introduced through the perimeter lumen, the air or liquid enters inflatable balloon 804 and inflates inflatable balloon 804. Inflatable balloons 804 may be deflated when air or liquid is aspirated from the perimeter lumen. Preferably, the at least one perimeter lumen may merge into a single connector (not shown) for synergistic inflation and deflation of the at least one inflatable balloon 804. Inflatable balloon 804 may have a diameter of between 20 mm and 50 mm, and preferably 30 mm, such that when inflatable balloon 804 wrapped around each of the at least one beam 807 is inflated, both the vein of the patient (not shown) and central lumen 824 are occluded, as illustrated in cross-sectional cut 809. In some embodiments, when deflated, inflatable balloons 804 are shaped as elongated sleeves wrapping the circumference of beams 807 and shaped such that when inflated would extend to an elongated tube shape. In some other embodiments, inflatable balloons 804 are designed such that when inflated, the inflatable balloons 804 form a round shaped extended circumference extending outward from tube 803 to occlude the vein of the subject completely. The occlusion is achieved through the inflation of the at least three inflatable balloons 804 which may initially take cylindrical shape and once additional liquid is introduced thereinto each inflatable balloon 804 increase in size to completely occlude the vein.

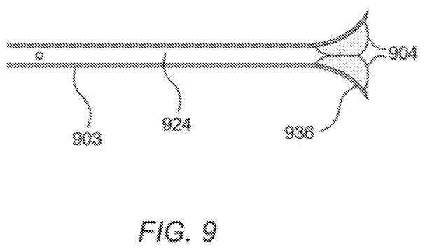

FIG. 9 schematically illustrates an alternative embodiment of inflatable balloons 904 on a cannula 900 adapted to occlude a vein and a central lumen of the cannula 900, in accordance with some embodiments of the present disclosure. Cannula 900 may comprise a tube 903 with a tip 936, the tube 903 enclosing a central lumen 924. Tip 936 may further comprise at least one inflatable balloon 904 positioned within central lumen 924. Inflatable balloon 904 may be a circular inflatable balloon that occludes central lumen 924 when inflated. Alternatively, there are two inflatable balloons 904 positioned substantially opposite to each other, such that the two inflatable balloons are 904 inflated, the surfaces of the two inflatable balloons 904 contact and compress against each other, thus occluding central lumen 924. In some embodiments, tip 936 may have a diameter between 2 cm to 3 cm and may be made of a super elastic material such that the tip 936 may expand when the at least one inflatable balloon 904 inflates. Non-limiting examples of super elastic materials are silicon and thermoplastic elastomers. In some embodiments, the at least one inflatable balloon 904 may be fluidly connected to one or more perimeter lumens (not shown) running longitudinally along tube 903 through two to four, and preferably four, openings (not shown) on each perimeter lumen (not shown).

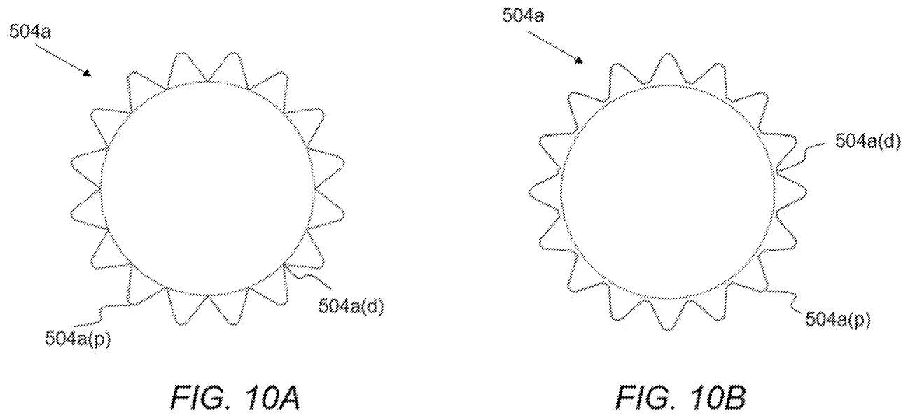

FIGS. 10A and 10B schematically illustrate an alternative embodiment of inflatable balloons 504a (when inflated) on cannula (not shown) in accordance with some embodiments of the present disclosure. The inflatable balloons may be serrated inflatable balloons. The serrated inflatable balloons may comprise serration teeth disposed on the surface of the balloons. The serration teeth may be provided in the form of strips embedded or protrusions disposed on the external surface of the inflatable balloons. The serration teeth may be outward protrusion (convex) 504a(p) or inward depression (concave) 504a(d) as illustrated in FIGS. 10A and 10B showing the cross-sectional cuts of the serrated inflatable balloons. It is to be understood that the size of protrusion/depression in FIGS. 10A and 10B is not shown to scale. The protrusion 504a(p) may be provided as a curved profile. The depression 504a(d) may be provided as a sharp (FIG. 10A), a flat (FIG. 10B) or a curved profile (not shown). The serration teeth of the serrated inflatable balloons may advantageously provide better fixation of the balloons when the same are inflated. Similar as previous embodiments, the serrated inflatable balloons may be coated with one or more layers of drug. In some embodiments, there may be 8 protrusions 504a(p) and 8 depressions 504a(d) disposed on the outer surface of the inflatable balloon. In some embodiments, there may be 16 protrusions 504a(p) and 16 depressions 504a(d) disposed on the outer surface of the inflatable balloon as illustrated in FIGS. 10A and 10B, respectively. In some embodiments, the size of the serration teeth, which can be represented by the height of the teeth, may be about 5% to 15% of the size of the inflatable balloon such as 5%, 6%, 7%, 8%, 9%, 10%, 11%, 12%, 13%, 14% or 15%. In some embodiments, when the size of the inflatable balloon is 30 mm, the height of the serration teeth may be about 1.5 mm to 4.5 mm, preferably about 2 mm, 3 mm or 4 mm. To have a better fixation of the balloons when inflated, the inflatable balloons may have a rugged (or uneven) outer surface where a plurality of protrusions/depressions is disposed on the outer surface of the inflatable balloons. The protrusions or depressions disposed may form a regular or random pattern.

It should be appreciated that the above-described apparatus may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled in the art that the present invention is not limited to what has been particularly shown and described hereinabove. Rather the scope of the present invention is defined only by the claims, which follow.

1. A cannula comprising:
a first tube having a first distal end for placement in a first vein of a patient;
a first central lumen extending longitudinally along the first tube;
at least one first hole positioned on the first distal end, the at least one first hole fluidly connected to the first central lumen;
at least one first peripheral lumen extending longitudinally along the first tube; and
at least one first inflatable balloon connected to the first tube, each of the at least one first inflatable balloon fluidly connected to one of the at least one first peripheral lumen.

2. The cannula of claim 1, wherein the at least one first inflatable balloon is adapted to occlude the first central lumen and the first vein of the patient when inflated.

3. The cannula of claim 2, wherein the at least one first inflatable balloon is positioned at a tip of the first tube within the first central lumen.

4. The cannula of claim 2, wherein the first tube comprises one or more tube segments, the one or more tube segments connected to each other at least one beam.

5. The cannula of claim 4, wherein each of the at least one first inflatable balloon is wrapped around each of the at least one beam.

6. The cannula of claim 1, wherein the at least one first inflatable balloon is wrapped around the first distal end of the tube, the at least one first inflatable balloon adapted to occlude the first vein of the patient when inflated.

7. The cannula of claim 1, further comprising a branch cannula, the branch cannula comprising:
a second tube having a second distal end for placement in a second vein of the patient;
a second central lumen extending longitudinally along the second tube, the second central lumen fluidly connected to the first central lumen;
at least one second hole positioned on the second distal end, the at least one second hole fluidly connected to the second central lumen; and
at least one second peripheral lumen extending longitudinally along the second tube;
at least one second inflatable balloon connected to the second tube, each of the at least one second inflatable balloon fluidly connected to one of the at least one second peripheral lumen.

8. The cannula of claim 7, wherein the at least one second inflatable balloon is wrapped around the second distal end of the second tube, the at least one second inflatable balloon adapted to occlude the second vein of the patient when inflated.

9. The cannula of claim 7, wherein the cannula is coated by one or more layers of drug.

10. The cannula of claim 9, wherein the drug is an anticoagulant drug, anti-inflammatory drug, anti-thrombogenic agent or mixture thereof.

11. The cannula of claim 7, wherein the at least one first inflatable balloon or the at least one second inflatable balloon is a serrated inflatable balloon.

12. A cannula comprising:
a tube having a distal end for placement in a first vein, an atrium, and a second vein of a patient;
a central lumen extending longitudinally along the tube;
at least one peripheral lumen extending longitudinally along the tube; and
a first and a second inflatable balloon connected to the tube, the first and second inflatable balloon each fluidly connected to one of the at least one peripheral lumen.

13. The cannula of claim 12, wherein the first inflatable balloon and the second inflatable balloon are interspersed with an intra-atrial region.

14. The cannula of claim 13, wherein the intra-atrial region is solid.

15. The cannula of claim 12, further comprising a third hole in the intra-atrial region, the third hole connected to one of the at least one peripheral lumen extending longitudinally along the tube.

16. The cannula of claim 12, wherein the size of the first and second inflatable balloon is the same.

17. The cannula of claim 12, wherein the distance between the first and second inflatable balloon is between 6 to 14 cm.

18. The cannula of claim 12, wherein the cannula is coated by one or more layers of drug.

19. The cannula of claim 18, wherein the drug is an anticoagulant drug, anti-inflammatory drug, anti-thrombogenic agent or mixture thereof.

20. The cannula of claim 12, wherein the first or the second inflatable balloon is a serrated inflatable balloon.
